SEQUENCE LISTING (1 sequence)

SEQ ID NO: 1 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

(19) United States
(12) Patent Application Publication
(10) Pub. No.: US 2008/0268476 A1
(43) Pub. Date: Oct. 30, 2008

(54) Nectin 4 (N4) as a Marker for Cancer Prognosis

(75) Inventor: Marc Lopez, Marseille (FR)

Correspondence Address: FOLEY AND LARDNER LLP;SUITE 500, 3000 K STREET NW, WASHINGTON, DC, 20007, US

(73) Assignee: Institut National De La Sante Et De La Recherche Medicale (INSERM), Paris (FR)

(21) Appl. No.: 11/596,040

(22) PCT Filed: May 12, 2005
(86) PCT No.: PCT/IB2005/001858

§ 371 Date: Jan. 10, 2007

(30) Foreign Application Priority Data
May 12, 2004 (EP) 04291210.5

(51) Int. Cl. G01N 33/53; G01N 33/574; C12Q 1/02

(52) U.S. Cl. 435/7.92; 436/64; 435/29

(57) ABSTRACT

The present invention relate for a method for prognosis cancer, in particular metastatic breast cancer comprising doing a dosage of Nectin 4, in a soluble form or in transmembrane form, in a sample, the presence of Nectin 4 being indicative of a cancer.

The present invention relates for a method for prognosis cancer, in particular metastatic breast cancer comprising doing a dosage of Nectin 4, in a soluble form or in transmembrane form, in a sample, the presence of Nectin 4 being indicative of cancer.

### BACKGROUND OF THE INVENTION

Improving methods allowing early-stages tumour detection and the following of tumour progression are probably among the major challenges in cancer therapy. Protein molecular tumour markers are commonly used in this respect, particularly in sera of patients. Even though serological markers are reliable and useful in prostate and colorectal cancers, for most other types of cancer either detection may be unreliable or no specific markers are yet available (1).

Considering for example breast cancer, it is one of the most common causes of cancer-related deaths in women. It affects approximately one million women per year. Despite improvements in diagnosis and treatment of this disease in the past decades, the survival rates remain low in comparison with other cancers.

Patients having breast cancer are presently identified by such means as mammography, fine needle aspiration biopsy (FNAB), FNAB guided by mammography, biopsy, magnetic resonance imaging (MRI), or other standard means that may include dosing a patient with radiation or incurring tissue damage in the process of getting a tissue sample to analyze. These methods are deficient because they do not detect early cancer, cannot detect precancer, and may cause damage to patients that have cancer by disrupting tissue near and around the cancerous lesion, and may also cause a serious risk of unclean margins after lesion removal. In addition, standard methods to screen for cancer such as mammography, FNAB, and biopsy also provide frequent opportunity for an ambiguous or false result. Thus, the medical community would benefit greatly from the application of a sensitive, non-radiation based, and non-invasive identification means for breast cancer, and a method to identify breast precancer.

At present, carcinoembryonic antigen (CEA) and CA 15-3 levels are commonly measured in sera and are more reliable in advanced disease than at early stages (2-4). These markers are helpful in following the course of patients with established cancer, especially to monitor response to therapy and to anticipate relapse. However these markers cannot detect all the patients with metastatic tumors and this evaluation is not accurate as 35% of patients with advanced breast cancer do not present any detectable levels of these markers.

Therefore, the purpose of the invention is to provide a diagnostic test that significantly improves detection of cancer by analysing a new marker named N4 (Nectin 4), in a soluble form or in transmembrane form.

Nectin 4 is a member of a new family of cell adhesion molecules named Nectins (13, 27-29).

Nectins are members of the immunoglobulin superfamily (IgSF) and are adhesion molecules that participate in the organization of epithelial and endothelial junctions and serve as receptors for herpes simplex virus entry. They are homologues of the poliovirus receptor (PVR/CD155), and were also named poliovirus receptor-related (PRR) proteins.

Four members have been described: PVR/CD155, Nectin1/PRR1/CD111, Nectin2/PRR2/CD112, and Nectin 3/PRR3 (5, 7, 9, 11). Their ectodomain is composed of three immunoglobulin (Ig)-like domains of V, C, C types and shares between 30 and 55% amino acid identity. Expression of Nectin/PRR molecules is generally broad in tissues, including hematopoietic, neuronal, endothelial, and epithelial cells, except for Nectin3, which displays a more restricted expression (5, 7, 9, 11, 13, 27, 28).

Nectin 1/PRR1 (also named herpes immunoglobulin receptor (HIgR) or herpesvirus entry (HveC)) serves as HSV entry receptor (13, 29). Nectin 1 appears to be the major HSV receptor as it mediates entry of all the HSV-1 and HSV-2 strains tested as well as of animal alphaherpesviruses. Nectin2/PRR2/(HveB) and PVR/CD155 (HveD) serve as receptor for a limited range of alphaherpesviruses (30, 31). Nectin1 and Nectin2 are involved in the cell to cell spreading of the virus (32).

Five Nectins have been described so far. All but Nectin 4 are expressed in epithelial, endothelial, hematopoietic and neuronal cells in adult tissues (10-14). Nectin 4 is mainly expressed during embryogenesis but is not detected in adult tissues (8). Nectin 4 is a natural ligand of Nectin 1 involved in the Cleft Lip/Palate Ectodermal Dysplasia (15, 16). During mouse development, Nectin 1 is expressed in oro-facial epithelia but no data are available concerning Nectin 4 expression in these tissues. Nectin 4 is a transmembrane adhesion molecule expresses during embryogenesis of aminoacids sequence SEQ ID N^{o} 1.

Nectin 4 (i) is structurally related to the Nectin family members; (ii) is expressed mainly in placenta in human tissues, presents a broader distribution in mouse tissues, and is expressed in mouse embryo; (iii) is a 66-kDa protein that co-localizes and interacts with the PDZ domain of afadin; (iv) recruits afadin at cadherin-based adhesion junctions; (v) is a Ca²^{⁺}-independent homophilic adhesion molecule; (vi) is a new ligand for Nectin1 but not for Nectin2, Nectin3, and PVR/CD155; and (vii) binds Nectin1 through the extracellular V domain interaction.

Nectin 4 expression is mainly restricted to endothelial cells in placenta and is expressed at embryonic days 11, 15, and 17 in mouse embryo. Previous reports have described the fundamental role of afadin in the organization of cell-cell junctions during mouse development (33, 34).

We have now found that Nectin 4, is present in a soluble form in the population afflicted with cancer, and is absent in the normal population, which makes it a new reliable diagnostic marker for cancer. More particularly a metastatic breast cancer. We also discovered that tumour cells of said subject express Nectin 4, in a transmembrane form.

Indeed, Nectin 4 is not expressed in normal breast epithelium but is found in 67% and 10% of ductal and lobular carcinomas respectively. Soluble form of Nectin 4 is naturally produced in vitro and circulating form of Nectin 4 is detected in 51% of sera (n=69) from subjects with metastatic breast tumor.

### DESCRIPTION

Therefore, in a first embodiment, the invention is aimed at an in vitro or ex vivo method for prognosis cancer comprising detecting the presence or the absence of Nectin 4 in a sample, the presence of Nectin 4 being indicative of cancer.

The term “detecting” or “detection” or “detect” include assaying, quantitating, imaging or otherwise establishing the presence or absence of cancer or Nectin 4, or assaying for, imaging, ascertaining, establishing, or otherwise determining one or more factual characteristics of cancer, metastasis, stage, or similar conditions. The methods can be used to detect the presence of cancer metastasis. They can further be used to monitor cancer chemotherapy and cancer reappearance.

The term “Nectin 4”, or “Nectin 4 protein” includes human Nectin 4 (N4), in particular the native-sequence polypeptide, isoforms, chimeric polypeptides, all homologs, fragments, and precursors of human Nectin 4. The amino acid sequence for native Nectin 4 include the sequences of GenBank Accession N^{o} AF426163 and shown in SEQ ID N^{o} 1.

The term “sample” and the like mean a material known or suspected of expressing or containing Nectin 4. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. It will be understood that the expression “method” comprise or exclude the step of obtaining said sample.

According to the present invention, Nectin 4 is detected by at least one anti-Nectin 4 antibody.

The method for using an antibody to detect expression of Nectin 4 protein in a sample comprises:
a) combining an antibody specific for Nectin 4 with a sample under conditions which allow the formation of antibody-protein complexes, and
b) detecting complex formation, wherein complex formation indicates expression of the protein in the sample. Expression may be compared with standards and is diagnostic of cancer.

The standard may correspond to levels quantitated for samples from control subjects without cancer, with a different stage, or from other samples of the subject, or any other negative control sample.

In particular, in the method according to the present invention, the sample is a subject's serum or plasma.

The term “subject” or “patient” refers to a warm-blooded animal such as mammal which is afflicted with or suspected to be afflicted with cancer. Preferably, “subject” refers to a human.

In the method according to the present invention, where the sample is a subject's serum, the cancer is a metastatic cancer, advantageously a metastatic breast cancer. In the method according to the present invention where the sample is a subject's serum, Nectin 4 is on a soluble form.

The method according to the present invention where the sample is a subject's serum further comprises dosing the level of soluble Nectin 4.

Advantageously, the presence of at least 30 pM of soluble Nectin 4 is indicative of a metastatic cancer, advantageously indicative of a metastatic breast cancer.

In the method according to the present invention, anti-Nectin 4 antibody is directed against the soluble form of Nectin 4, and more particularly against the ectodomain of Nectin 4.

In the method according to the present invention where the sample is a subject's serum, the presence of soluble Nectin 4 is determined by Immuno Assay.

Examples of such assays are radioimmunoassay, enzyme immunoassays (e.g. ELISA), immunofluorescence, immunoprecipitation, latex agglutination, hemaglutination, and histochemical tests.

Advantageously, the presence of soluble Nectin 4 is determined by an Enzyme-Linked Immuno Sorbent Assay (ELISA).

In a preferred embodiment, the antibody used for detecting or dosing the soluble Nectin 4 in a serum sample is an antibody directed against the soluble Nectin 4, particularly capable of specifically recognizing the soluble form of Nectin 4, more particularly directed against the ectodomain of Nectin 4.

In particular, in the method according to the present invention, the sample is subjects' tumor cells.

The method according to the present invention, where the sample is subjects' tumor cells, further comprises detecting the signal and determining the ratio of cell-expressing Nectin 4.

Advantageously, a quick score (percentage of cells expressing Nectin 4× labeling intensity) above 10 is indicative of a cancer, advantageously indicative of a metastatic cancer, more advantageously indicative of a metastatic breast cancer.

The labeling intensity has to be understood as 1 for a weak intensity, 2 for a medium intensity, and 3 for a strong intensity.

A weak signal is a signal above the background, a strong signal is a signal similar to a control antibody that detect a strongly expressed antigen. A medium signal is a signal between a weak signal and a strong signal.

The detection of Nectin 4 expression in tumor cells can be performed with any methods currently employed by the man skilled in the art.

For example, one can detect the presence of Nectin 4 by immunohistochemistry.

The antibody may be used in histochemical analyses, for example, at the cellular and subcellular level, to detect Nectin 4 protein, to localize it to particular tumor cells and tissues, and to specific subcellular locations, and to quantitate the level of expression.

It basically consists of staining tissue section with the antibodies against Nectin 4. The antibodies according to the invention are, for example, monoclonal or polyclonal antibodies or Fab or F(ab′)2 fragments thereof. They may also be in the form of immunoconjugates or of labelled antibodies (immunofluorescence, gold labelling, enzymatic immunoconjugates) so as to obtain a detectable and/or quantifiable signal (35). Alternatively, if a non labelled mouse Nectin 4 antibody is used, the method further comprises a step consisting of incubating with an antimouse immunoglobulin coupled with a label (fluorescent or enzymatic for example). For example, it is possible to use a biotinyl goat antimouse immunoglobulin and the detection is performed by incubating with the streptavidin biotin peroxydase complex and its substrate.

Thus, the method of the invention can include the steps of:

a) Rinsing slides in TBS (for few minutes)
b) Removing excess liquid from around specimen
c) Applying normal serum to cover specimen and incubating
d) Tapping off serum
e) Applying appropriate quantity of enzyme-conjugated primary anti-Nectin 4 antibody
and incubating
   f) Applying substrate-chromogen solution and incubating until the desired color intensity has developed.

Alternatively, step e) may consist of applying primary anti-Nectin 4 antibody and incubating, repeat step a) and b); followed by step f) which is applying enzyme-conjugated secondary antibody directed against primary antibody immunoglobulin and g) applying substrate-chromogen solution and incubating until the desired color intensity has developed.

The method according to the present invention comprises the use of a labeled anti-Nectin 4 antibody.

In particular, said labeled antibody is a fluorescent, gold or enzyme immuno-conjugate.

Moreover, the method according to the present invention comprises the use of an anti-Nectin 4 primary antibody and a staining with a labeled second reagent antibody directed against said primary antibody.

Advantageously, the method according to the present invention comprises besides detecting the presence or the absence of other cancer markers.

Other markers include but are not limited to a member of the HER family of receptor tyrosine kinases, estrogen receptors, interleukins, cadherins (e.g. E-cadherin), BRCA1, BRCA2, CA125, CA15-3, CA19-9, and carcinoma embryonic antigen (CEA).

Advantageously, other markers are carcinoma embryonic antigen (CEA) and CA 15-3.

Interestingly, we have showed that the association of Nectin 4 with carcinoembryonic antigen (CEA) and CA15.3 markers increased the detection from 71% to 84%. Nectin 4 serum levels increase during disease progression. Altogether our results emphasize that Nectin 4 is a new valuable marker for metastatic breast cancer and open new alternatives in subjects that present undetectable levels of CEA and CA15-3.

The present invention also relates to a method for medical imaging a tumor from a subject comprising:
a) incubating the tumor with a labeled anti-Nectin 4 antibody for a sufficient period of time to permit the antibody to react with Nectin 4;
b) detecting the presence of the label localized to the tumor.

The present invention also relates to the use of a labeled anti-Nectin 4 antibody for the preparation of a composition for medical imaging a tumor from a subject, particularly a metastatic tumor and more particularly a metastatic breast tumor.

In a second embodiment, the invention is aimed at a method for prognosis cancer comprising detecting Nectin 4 level in a subject sample and determining the level of Nectin 4 compared to the level in a control sample, a significant level in a subject sample being indicative of a poor outcome.

The expression “poor outcome” is meant to refer herein to a shorter overall mean survival rate compared to the overall cancer population, in particular breast cancer population.

The term “significant level” is meant to refer to a level above 30 pM.

The invention also relates to a method for the therapeutic follow-up of an anticancer treatment of a subject characterized in that the presence or the absence of Nectin 4 is detected during or after the treatment.

In particular, the absence or the decrease of Nectin 4 is significant of a positive response to the treatment.

In particular, in the method for prognosis cancer according to the present invention, the sample is a subject's serum.

In the method for prognosis cancer where the sample is a subject's serum, according to the present invention, Nectin 4 is on a soluble form.

In particular, in the method for prognosis cancer according to the present invention, the sample is subject's tumor cells.

In the method for prognosis cancer, according to the present invention the expression of Nectin 4 in a sample is evaluated by combining quantitative RT-PCR and linear discriminant analysis, competitive quantitative PCR.

It is also possible to use cDNA micro-array technology (36). The method here is to quantitatively analyze fluorescence signals that represent the relative abundance of mRNA coding for Nectin 4 from two distinct tissue samples. Two different samples of mRNA (one normal sample control and one from the subject can be labelled with different fluorescent molecules and then co-hybridized on to arrayed Nectin 4 gene. Ratios of gene-expression levels between the samples are calculated and used to detect meaningfully different expression levels between the samples (U.S. Pat. No. 6,245,517). Other examples include high density tissue microarray technology involving arraying up to thousands of cylindrical tissue cores from individual tumors on a tissue microarray (37). This technology allows rapid analysis of a large number of samples so that the statistical relevance is determined in a single experiment. Arrays have been made containing different tumor types (38) and multiple stages and grades within one tumor type (39, 40). This technology is now considered useful for rapidly characterizing the prevalence and prognostic significance of differentially expressed genes identified using cDNA array technology. Tissue microarrays have also been useful to study the expression patterns of putative tumor suppressor genes (41).

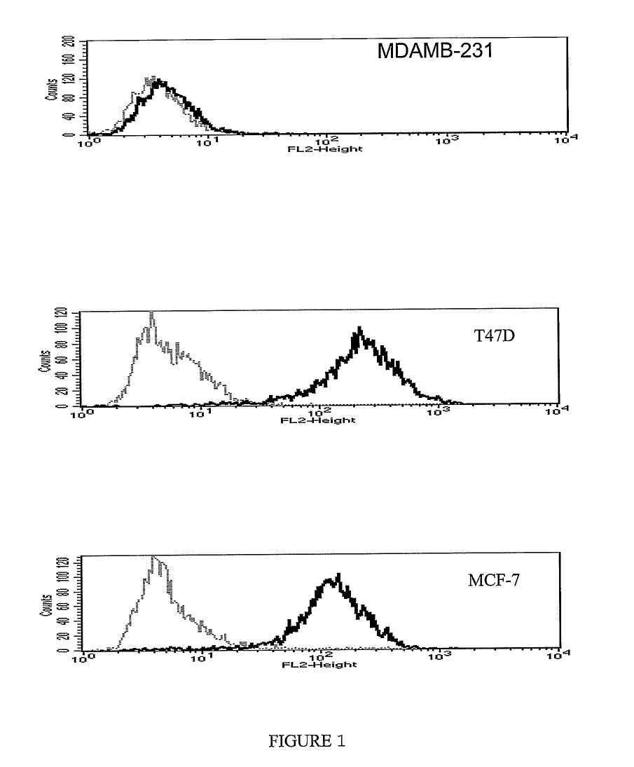

It is also possible to use gel electrophoresis for detecting Nectin 4 in a sample. For example, the invention encompasses a method for detecting Nectin 4 level in a breast tissue sample comprising 2D-gel electrophoresis and Mass Spectrometry, in particular Surface-enhanced laser desorption and ionisation time of flight (SELDI-TOF) Mass Spectrometry. Here, the purpose is to obtain proteomic profiling of normal sample versus Nectin 4 positive breast cancer sample so as to directly detect the level of Nectin 4 expression with such profiles. In this regard, mass spectrum are obtained from test samples, which generate signature patterns (plot relative abundance of key discriminatory proteins including Nectin 4). General process for pattern discovery and pattern matching are described in Petricoin F, Use of proteomic patterns in serum to identify ovarian cancer, The Lancet, Vol. 359, Feb. 16, 2002 (42); Zhao Rui et al, Use of serological proteomic methods to find biomarkers associated with breast cancer, Proteomics, Vol. 3, Issue 4, p 433-439, 2003 (43). A diagram representing this method is illustrated at FIG. 1 of Sandy Kennedy, Toxicology Letters 120 (2001) 379-384 (44), incorporated herein in the description. It is further envisioned to profile fluids proteins from breast cancer Nectin 4+, Nectin 4− subjects and also control sample to identify surrogate fluids makers.

In a third embodiment, the invention is aimed at a kit for performing the method according to the present invention, comprising either a labelled anti-Nectin 4 or a first anti-Nectin 4 antibody and a second labelled anti-Nectin 4 antibody.

In another aspect, the kit can comprises the primers for specifically amplifying Nectin 4 mRNA or cDNA, such as primers for performing q-RT-PCR for example and/or a Nectin 4 c-DNA array. In this regard, specific primers and probes can be designed for example by referring to the sequence of GenBank accession number AF426163. The primers herein are selected to be “substantially” complementary to the above DNA sequence. This means that the primers must be sufficiently complementary to hybridize under stringent conditions with their respective strands. Therefore, the primer sequence need not reflect the exact sequence of the template. For example, a non-complementary nucleotide fragment can be added to the 5′ end, with the remainder of the primer sequence being complementary to the strand. Also, longer sequences can be interspersed into the primer, provided that the primer sequence has sufficient complementary with the sequence to hybridize therewith and form the template for synthesis of the extension product. One type of quantitative PCR assay involves simultaneously amplifying control DNA which amount is known and samples suspected to contain a target sequence. Following amplification, the amounts of amplified products (amplicons) are compared (45, 46). In other methods, the control molecule is similar to the target Nectin 4 mRNA (quantitative-competitive PCR (QC PCR)). Following competitive amplification, the two products synthesized (amplicons) are distinguished, for example, by size using gel electrophoresis (47)

A more recently developed type of quantitative PCR assay is the 5′-nuclease assay and “real-time PCR.” (48, 49, U.S. Pat. No. 5,538,848). This method is based on probes that are DNA sequences labeled with two different fluorescent dyes, for example, a reporter dye and a quenching dye. Kits from the Applied Biosystems are available under the trademark TaqMan™ and fluorescence can be monitored throughout the PCR amplification with the Applied Biosystems ABI PRISM 7700 for example. Thus, the kit of the invention may comprise pre-labelled primers and optionally reagents as described in the above documents for q-PCR, QC-PCR and real-time PCR.

In a fourth embodiment, the invention is aimed at a kit comprising a first anti-Nectin 4 antibody and a second anti-Nectin 4 antibody, said first and second antibody being directed against different Nectin 4 epitopes and wherein the binding of the first antibody does not interfere with the binding of said second antibody.

In a fifth embodiment, the invention is aimed at the use of a method according to the present invention and/or kit according to the present invention for the prognosis of patients afflicted with cancer, advantageously metastatic cancer, more advantageously metastatic breast cancer. It also relates to the use of a method and/or kit for the initiation of adequate therapy early in the cause of the disease, for providing an ex vivo assessment of the antitumor effects of the chemotherapy in the course of the therapy.

The “therapy” may be any therapy for treating cancer, including but not limited to therapeutics, radiation, immunotherapy, gene therapy and surgical removal of tissue. Therefore, the method and/or kit can be used to evaluate a subject before, during, and after therapy.

Thus, the invention is aimed at the use of a method or kit as defined above as a predictor of cancer prognosis and survival, advantageously a predictor of breast cancer prognosis and survival.

Indeed, this is the first time that Nectin 4 level of protein expression is correlated to cancer prognosis and survival, more particularly, to breast cancer prognosis and survival.

In a sixth embodiment, the invention is aimed at antibody wherein said antibody is a monoclonal, polyclonal or a fragment thereof, specifically directed against Nectin 4. In seventh embodiment, the invention is aimed at production process of the antibody of the present invention wherein
Antibodies are produced by hybridomas of IgG1 k isotype.
After adaptation of said hybridomas to Serum-free medium, (for example hybridoma-SFM, 12045-076 from Invitrogen) conditioned medium are harvested, centrifuged, filtered through a filter (for example 0.45 μm filter, Amicon).
Antibodies purification is performed by affinity column chromatography (for example, Affigel ProteinA, BioRad) using a binding buffer (for example MAPSII binding buffer, BioRad)
Antibodies are eluted with an acetate buffer pH 3.0 and neutralizes in a Tris-Hcl buffer pH 8.8
Antibodies are dialysed twice in PBS buffer (for example Invitrogen)
Antibodies are concentrated using a Centricon (Amicon) cartridge with a 10 kDa molecular weight cutoff membrane
Antibodies purity is assessed by SDS PAGE analysis and coomassie blue staining
Concentration is determined by calorimetric Bradford assay (for Example BioRad)
Antibodies are titrated by fluorescent staining of Nectin 4 expressing cells

According to this process, the yield may be approximately 1 mg of Antibodies/Liter.

In an eight embodiment, the invention is aimed at a kit for medical imaging comprising labeled anti Nectin 4 antibody according to the present invention, or the antibody obtainable by the process according to the present invention.

According to a particular aspect of the invention, an in vivo method for imaging cancer is provided comprising:
a) injecting a patient with an anti-Nectin 4 antibody, the antibody carrying a label for imaging the cancer
b) allowing the antibody to incubate in vivo and bind to Nectin 4 associated with the cancer, and
c) detecting the presence of the label localized to the cancer.

Examples of labels useful for imaging are radiolabels, fluorescent labels (e.g. fluorescein and rhodamine), nuclear magnetic resonance active labels, positron emitting isotopes detectable by a positron emission tomography (“PET”) scanner, chemiluminescers such as luciferin, and enzymatic markers such as peroxydase or phosphatase. Short-range radiation emitters, such as isotopes detectable by short-range detector probes can also be employed.

In a ninth embodiment, the invention is aimed at the use of the kit for medical imaging according to the present invention or labeled anti Nectin 4 antibody according to the present invention or the antibody obtainable by the process according to the present invention for preparing a product for medical imaging of tumor cells, advantageously metastatic cells.

The invention is further described in the examples and figures below. It should be understood that the specific examples while indicating preferred embodiments of the invention are given by way of illustration only.

### LEGENDS

FIG. 1: Analysis of Nectin 4 expression in normal and tumor cells. Nectin 4 expression level was monitored with the anti-Nectin 4 N4.61 monoclonal antibody (mAb) (black line) and compared with a mouse irrelevant IgG1 (gray line), both used at 5 μg/ml.

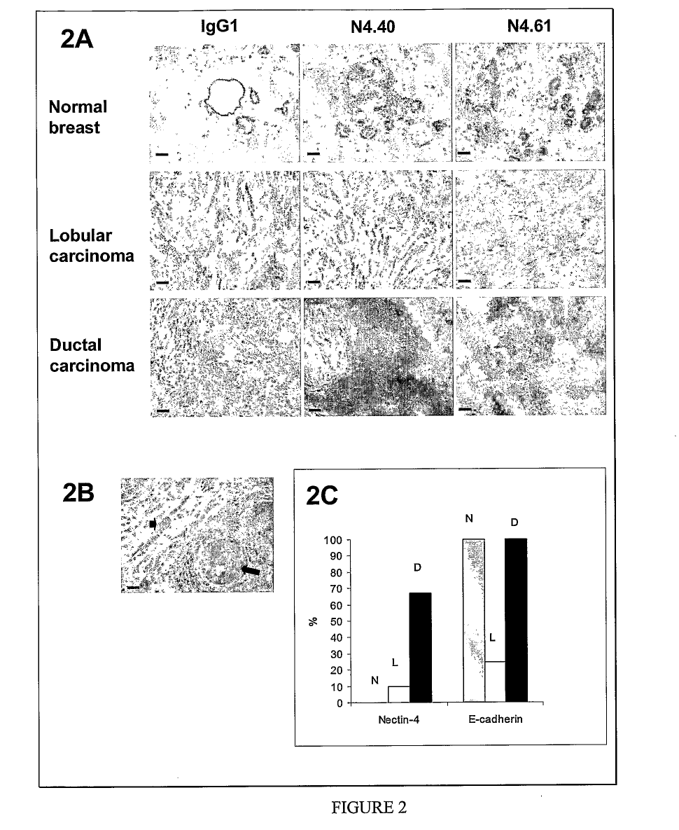

FIG. 2: Nectin 4 expression in breast carcinomas. Immunostaining procedure of Nectin 4 was described in the methods section using the two different anti Nectin 4 mAbs N4.40 and N4.61. A: Representative results obtained from normal and tumor breast samples. In 100% of cases, normal breast epithelium did not express Nectin 4. It is of note that myoepithelial and stromal cells did not express Nectin 4. In 90% of cases both in situ and invasive lobular carcinoma did not express Nectin 4 at all. Nectin 4 expression was found in 67% of ductal carcinomas. Expression was generally strong in all the tumor cells of the section. Bar 50 μm. Magnification: ×200. B: Top: Both invasive (arrowhead) and in situ (arrow) components of ductal carcinoma were immunostained by Nectin 4. Magnification: ×200. In all cases Nectin 4 immunostaining was prominently cytoplasmic. Bottom: Imnunofluorescence studies on breast tumor sections revealed Nectin 4 cytoplasmic expression (arrowhead) but also highlighted a junctional staining (arrow). Magnification: ×640. C: Nectin 4 and E-cadherin expression in normal breast tissues (n=5) (gray bar), lobular (n=31) (white bar), and ductal carcinomas (n=27) (black bar).

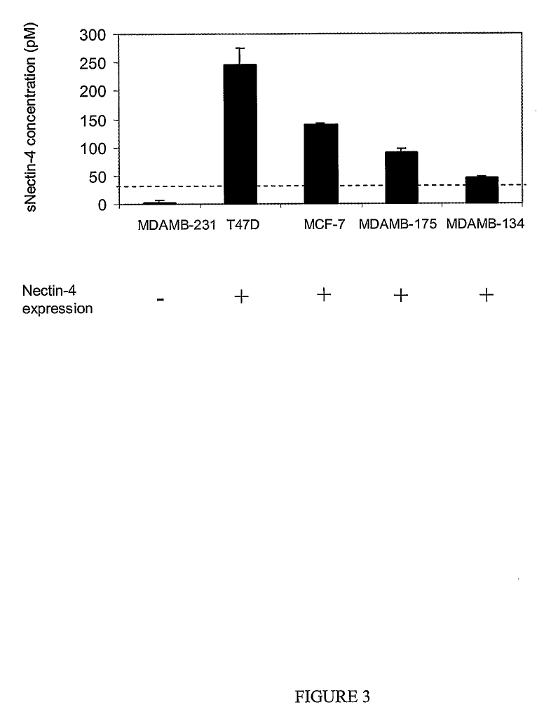

FIG. 3: Production of a soluble form of Nectin 4 in vitro. One hundred μl of a 3 day culture medium of breast tumor cell lines was analyzed in duplicate by ELISA as described in the methods section. Concentration was deduced from a titration analysis of a soluble recombinant Nectin 4-Fc protein. Threshold was indicated and corresponds to 30 pM. Cell lines were selected on the basis of Nectin 4 expression.

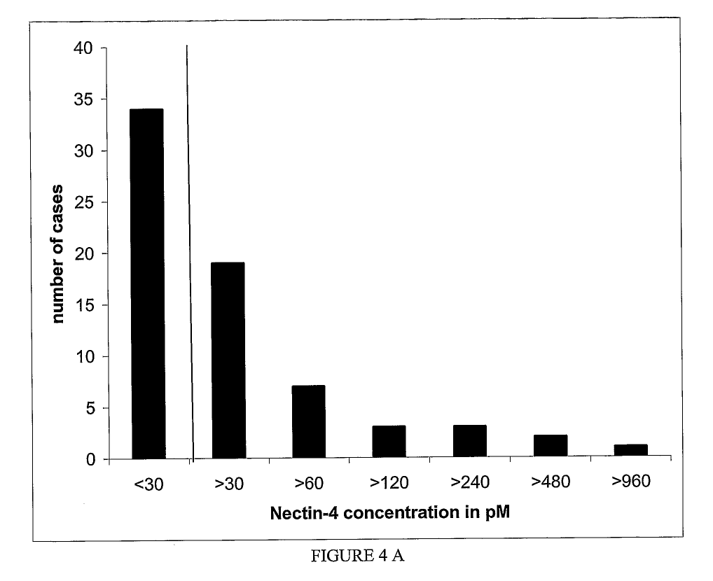

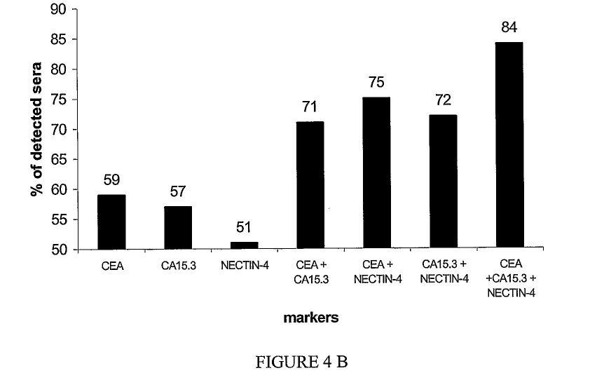

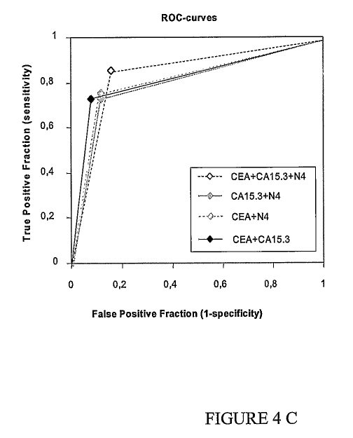

FIG. 4: A soluble form of Nectin 4 represents a new serum marker of metastatic breast carcinomas. One hundred μl of serum was analyzed in duplicate by ELISA as described in the methods section. A: The histogram summarizes the various levels of soluble Nectin 4 detected in the sera of 69 patients with a metastatic breast carcinoma at diagnosis. B: Comparative analysis of soluble Nectin 4, CEA, and CA15.3 markers in these patients. Markers were analyzed individually or in association, and the frequency corresponds to the percentage of sera that can be detected with one, two or three of these markers. C: Receiving Operator Characteristic (ROC) curves were calculated to estimate the accuracy of the association of these markers in breast cancer diagnosis.

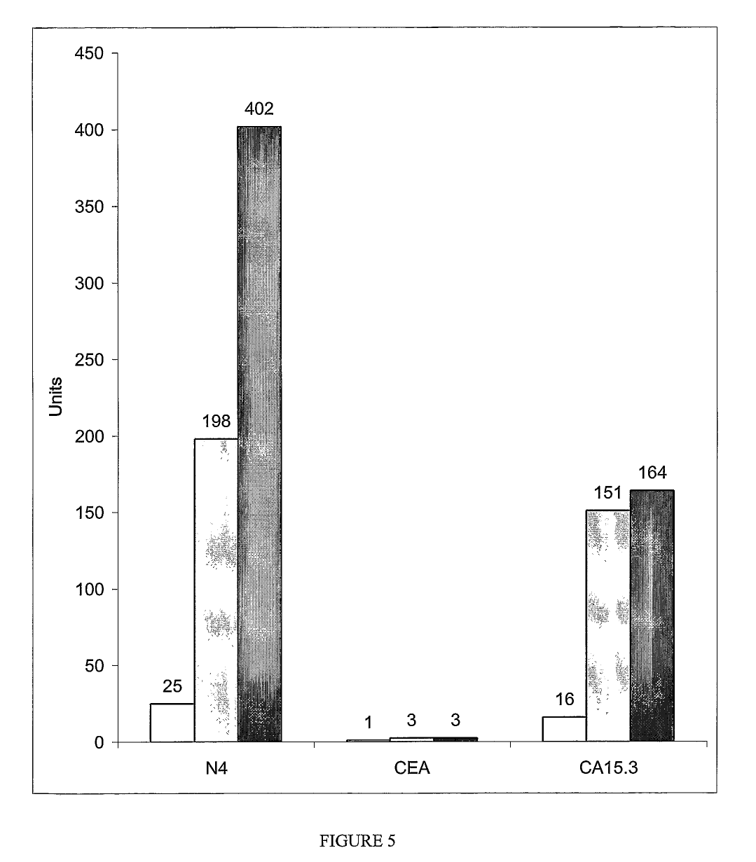

FIG. 5: The serum Nectin 4, a useful marker to monitor disease progression. Soluble Nectin 4, CEA and CA15.3 serum levels were determined in a patient with a ductal carcinoma that expresses Nectin 4. The three markers were not detected at the time of diagnosis (white bar). During the progression of the disease, 32 (gray bar) and 39 months (black bar) after diagnosis, Nectin 4 serum levels increased to reach 402 pM. Ordinate represents pM for Nectin 4 and International Units for CEA and CA15.3.

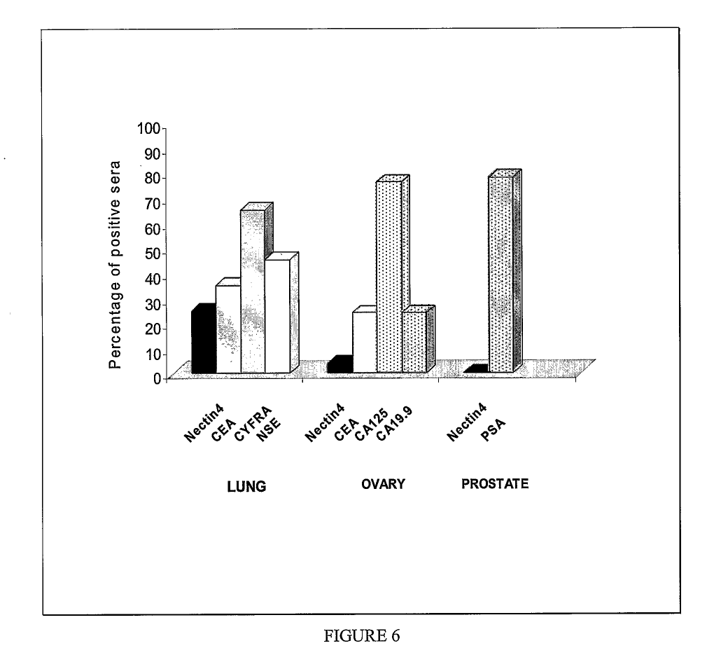

FIG. 6: Detection of soluble Nectin 4 in the sera of patients with other neoplasms. Serum obtained from lung, ovary and prostate cancers were tested for the presence of soluble Nectin 4 and compared with the commonly used markers. The frequency corresponds to the percentage of sera that can be detected with each one of these markers.

### EXAMPLE 1

### Methods

Cells and culture conditions. Human leukocytes were purified from healthy donors using ficoll separation. HUVEC were isolated and cultivated as previously described (19). CD34 positive cells were purified with MACS as already reported (12). Hematopoietic cell lines were cultivated in RPMI medium supplemented with 10% foetal calf serum. Adherent cell lines were cultivated in Dulbecco's modified Eagle's medium 45% CHO medium, supplemented with 10% foetal calf serum. Breast carcinoma cell lines were cultivated in 45% Dulbecco's modified Eagle's medium 45% CHO medium, supplemented with 10% foetal calf serum. Cells were purchased from ATCC (Manassa, Va.). Cells were cultivated in an air-5% CO2 atmosphere at constant humidity. Penicillin (50 U/ml), streptomycin (50 μg/ml) and glutamine (2 mM) were added in the different medium.

Breast tumor sample selection. A panel of 58 tumor samples prior any adjuvant therapy was obtained from women treated at the Institut Paoli-Calmettes. Tumors were classified according to the WHO classification; the histoprognostic grade used was the modified Scarf Bloom Richardson (SBR) grading for invasive lesions. There were 27 ductal, 31 lobular and 1 medullary carcinomas.

Serum selection. Panels of 45 sera from healthy donors, 53 sera from patients with non metastatic breast carcinomas at diagnosis, 70 sera from patients with metastatic breast carcinomas at diagnosis, 20 sera from patients with lung carcinomas, 25 sera from patients with ovary carcinomas, 23 sera from patients with prostate carcinomas were collected and included in this study.

Antibodies. Anti-Nectin 4 monoclonal antibodies were obtained after mice immunization with 20 μg of the recombinant soluble Nectin 4. Nectin 4-Fc protein were produced and purified as previously described (8). After screening on Cos cells expressing Nectin 4, two mAbs were isolated and named N4.40 and N4.61.

Immunohistochemistry (IHC). For Nectin 4, IHC was carried on five-μm sections from frozen tissue. Sections was fixed in acetone for 10 min and air dried for 10 min and rehydrated in TBST (Dako, Coppenhagen, Denmark). Staining was done at room temperature and DAKO EnVision™ System was used with Alkaline Phosphatase (AP) method. Slides were first incubated with the primary antibody N4.61 or N4.40 at 0.5 μg/ml for 30 min. After washes in TBST, slides were incubated with AP labelled polymer which is conjugated to secondary antibodies for 30 min. Fast Red substrate-chromogen solution was prepared and used as dye. Slides were counter-stained with hematoxylin, and coverslipped using Aquatex (Merck, Darmstadt, Germany). For E-cadherin, IHC was carried on 5-μm sections of formalin-embedded tissue specimens. They were deparaffinized in histolemon (Carlo Erba Reagenti, Rodano, Italia) and rehydrated in graded alcohol. Antigen enhancement was done by incubating the sections in citrate acid buffer pH6 (Dako, Coppenhagen, Denmark) as recommended. Slides were then transferred to a Dako autostainer. Staining was done at room temperature as follows: after washes in phosphate buffer, followed by quenching of endogenous peroxidase activity by treatment with 0.1% H2O2, slides were first incubated with blocking serum (Dako) for 10 min and then with the primary antibody anti-E-cadherin (1/2000, clone 36, Transduction Laboratories) for one hour. After washes, slides were incubated with biotinylated antibody against rabbit Ig for 20 min followed by streptavidin conjugated peroxidase (Dako LSABR2 kit). Diaminobenzidine was used as the chromogene, counterstained with hematoxylin, and coverslipped using Aquatex (Merck, Darmstadt, Germany) mounting solution. Slides were evaluated under a light microscope. Immunoreactivities were classified by estimating the quick score (Q) as previously described (20).

Cell surface expression analysis of Nectin 4. 2×105 cells were incubated for 60 min at +4° C. with 10 μg/ml of N4.40 or N4.61 mAbs, washed, and then revealed by incubation for 45 min at +4° C. with a phycoerythrin labelled goat anti-mouse antibody (Immunotech, France). Samples were processed by FACS analysis.

ELISA. A sandwich enzyme-linked immunosorbent assay was used to detect soluble Nectin 4 in conditioned culture medium. Ninety-six-well trays were coated with anti Nectin 4 N4.40 at 10 μg/ml. After saturation of wells with phosphate-buffer-saline containing 1% bovine serum albumin, 100 μl of culture medium or serum was incubated with 2.5 μg/ml biotinylated mAb N4.61 followed by streptavidin-peroxidase and One Step ABTS (Pierce). Optical density was read at 405 nm. We analyzed duplicates and reported the medium value.

Receiving Operator Characteristic (ROC) analysis. To determine the accuracy of an approach of diagnosis using the CEA, the CA15.3 and the Nectin 4 markers, receiver operating characteristic (ROC) curves were produced for the four possible associations: CEA+CA15.3+Nectin 4, CA15.3+N4, CEA+N4, CEA+CA15.3. Fifty one sera of primary breast carcinoma patients and sixty nine sera of metastasis breast carcinoma patients were considered for this study. Sensitivity, specificity and areas under receiver operating characteristic (ROC) curves were calculated in each case.

### Results

### Nectin 4 Expression in Breast Tumor Cell Lines:

We previously cloned human and murine Nectin 4. Northern blot analyses showed that Nectin 4 is expressed in mouse embryo from day 11 d.p.c. (8). In adult tissues, expression of Nectin 4 differs between mouse and human. In mouse, Nectin 4 is expressed in brain, lung, and testis. In human, Nectin 4 expression was only found in placenta and slightly in trachea among 23 tissues tested.

To extend this study we developed anti-Nectin 4 monoclonal antibodies (N4.40 and N4.61). We present here the results obtained on either primary cells or a panel of tumor cell lines of human origin (FIG. 1 and table 1).

Tables 1 show that we controlled that trypsin treatment did not affect cell surface expression of Nectin 4 (data not shown). Cell surface expression of Nectin 4 was assessed by FACS analysis. (−) No Nectin 4 expression, (+) Nectin 4 expression.

TABLES 1
Normal Cells

| | |
|---|---|
| endothelium | |
| ECRF | − |
| HuVEC | − |
| HbMEC | − |
| EAhy926 | − |
| epithelium | |
| HME | − |
| hematopoietic cells | |
| CD34+ | − |
| Monocytes | − |
| PMN | − |
| T lymphocytes | − |
| B lymphocytes | − |
| Masts | − |

Tumor cell lines

| | |
|---|---|
| Leukemia | |
| AML M4 | − |
| AML M5 | − |
| HL60 | − |
| TF1 | − |
| U937 | − |
| THP1 | − |
| JA16 | − |
| Carcinoma | |
| PC3 | − |
| DU145 | − |
| LNCAP | + |
| MiaPaCa | − |
| IMR-G | − |
| IMR-32 | − |
| IMR90 | − |
| A431 | + |
| Bewo | + |
| Tera1 | − |
| A704 | − |
| GCT | − |

Breast tumor cells

| | |
|---|---|
| benign | |
| HBL100 | − |
| MCF10F | − |
| MCF10A | − |
| malignant | |
| T47D | + |
| MCF-7 | − |
| Cama1 | + |
| MDA-MB-175 | + |
| MDA-MB-453 | + |
| SUM 225 | + |
| MDA-MB-137 | + |
| MDA-MB-134 | + |
| HCC1937 | + |
| HCC1187 | + |
| SUM 52 | + |
| MDA-MB-231 | − |

Nectin 4 is not expressed at the surface of normal hematopoietic cells, endothelial cells, and epithelial cells. Leukemic, lung, pancreas, neuroblastoma, kidney and colon tumor cell lines do not express Nectin 4. We found that Nectin 4 is expressed in the prostate carcinoma cell line LNCAP but not in PC3 and DU145. Nectin 4 is expressed in the epidermoid carcinoma cell line A431 and the choriocarcinoma cell line BeWo.

We tested 15 different breast cell lines. Three were derived from benign tumors and do not express Nectin 4, and 11 out of 12 were derived from malignant breast carcinoma and express Nectin 4. Even though this study is not exhaustive, we can conclude that Nectin 4 is expressed in malignant tumor cells and absent from normal cells as suggested by our previous study. In addition, our results highlight that Nectin 4 can be expressed at various levels in malignant breast carcinoma cell lines but not in benign or normal breast epithelial cells.

### Nectin 4 Expression in Breast Carcinoma Samples

Ductal and lobular carcinomas account for almost 80% and 15% of breast cancers, respectively. The two histologic types were analyzed by immunohistochemistry for Nectin 4 expression.

As expected from cell line analyses, Nectin 4 was not detected in sections of normal breast tissues. Interestingly, Nectin 4 was mainly expressed in sections of ductal breast carcinomas (FIG. 2A). Nectin 4 was expressed in both compartments of non invasive ductal carcinomas with an infiltrating component (FIG. 2B). A detailed analysis showed that Nectin 4 is exclusively expressed in carcinoma cells and absent from myo-epithelial and stromal cells (data not shown). Immunostaining highlighted a prominent cytoplasmic localization of Nectin 4 and a faint membrane staining (FIGS. 2A, 2B). This was not expected as Nectin 4 was detected at the cell surface of tumor cell lines by FACS analysis (FIG. 1), and Nectin 4 was previously described at E-cadherin-based adherens junctions in epithelial cells (8). However, even though immunofluorescence studies revealed cytoplasmic expression of Nectin 4, they also revealed a clear localization at intercellular junctions between carcinoma cells providing evidence that Nectin 4 is also expressed at the cell surface (data not shown).

Only 10% of lobular carcinomas expressed Nectin 4 vs 67% for ductal carcinomas (FIG. 2C). E-cadherin expression was analyzed in these samples: E-cadherin was detected in 25% of lobular carcinomas and 100% of ductal carcinomas in accordance with results describing frequent loss of expression of E-cadherin in lobular carcinomas (21-23). Our results show that expression of Nectin 4 is found de novo in ductal carcinomas but is frequently absent in lobular carcinomas. Nectin 4 was detected in one medullar breast carcinoma. Nectin 4 expression was not correlated with either histo-pronostic grade or axillary lymph node metastasis status (data not shown). Our data show that Nectin 4 is a new specific marker for breast carcinomas and its expression strongly correlates with histological type. In contrast to E-cadherin expression which is present in normal cells but lost in lobular carcinomas, Nectin 4 is not present in normal cells but is up-regulated in ductal carcinomas.

### Nectin 4 is a New Serological Marker in Metastatic Breast Carcinoma:

Two recent reports showed that Nectin-1 is cleaved by a protease activity (17, 18). In one case, this shedding leads to the release of Nectin-1 ectodomain in cell culture medium of Nectin-1 transfected cells (18). Since Nectin 4 is expressed in breast tumor cell lines, we tested the release of Nectin 4 in culture medium by ELISA. Levels that do not exceed 30 pM of recombinant Nectin 4, which represents the detection threshold, were considered as negative. No detectable soluble Nectin 4 was detected in the medium from the MDAMB-231 cell line that does not express Nectin 4. However, a soluble form of Nectin 4 was detected in the medium from the four cell lines expressing Nectin 4 at the cell surface with a concentration ranging from 50 pM to 240 pM (FIG. 3). Interestingly, among the cell lines tested, the T47D cell line that expressed the highest level of soluble Nectin 4, exhibits the highest level of cell surface Nectin 4. These results point out for the first time that soluble form of Nectin 4 can be constitutively produced by breast tumor cell lines. Circulating forms of adhesion molecules have been reported in different diseases especially in cancer. However, only two serum markers (CEA and CA15-3) are commonly used in the following of metastatic breast cancer but they fail to detect all patients. Since Nectin 4 is expressed in breast tumors and is released in culture medium, we thus looked for the presence of a circulating form of Nectin 4 in sera from patients with breast carcinoma. Using ELISA, we firstly found that soluble form of Nectin 4 was undetectable in 44 out of 45 normal sera. Nectin 4 was then investigated in 53 sera of patients with non metastatic breast cancer at diagnosis. Two and three patients were positive for CEA and CA15.3 markers respectively. Five sera presented Nectin 4 levels above the threshold and among them, one sera was also found positive for CEA and CA15.3 markers (data not shown).

We extended the analysis to a panel of sera of patients with metastatic breast tumors at diagnosis and compared with the level of CEA and CA15.3 markers. As shown in FIG. 4A, circulating forms of Nectin 4 were detected at various levels ranging from >30 pM to >1000 pM in 51% of tested sera (n=69). In these same samples, CEA and CA15.3 markers were detected in 59% and 57% of sera, respectively. Interestingly, whereas the combination of the two latter markers reaches 71% of detection, the association of CEA, CA15.3 and Nectin 4 allowed the metastasis detection of 84% (FIG. 4B). In other terms, 9 sera negative for both CEA and CA15.3 were positive for Nectin 4. The sensitivity, assessed by ROC curves, significantly increases from 0.72 (CEA+CA15.3) to 0.85 (CEA+CA15.3+Nectin 4), with a moderate loss of specificity (from 0.92 to 0.84) (FIG. 4C). Sensitivity and specificity for CEA+Nectin 4 and CA15.3+Nectin 4 were similar to CEA+CA15.3. This study shows that the combination of the three markers more accurately correlates with the metastatic status of the patient than either association of two markers. This correlates with previous results showing detection of soluble Nectin 4 in sera negative for CEA and CA15.3. This association of CEA, CA15.3 and Nectin 4 should be tested in clinical practice.

One of the major challenge of tumor marker is to anticipate the appearance of metastases. Even though our study is retrospective, we show that Nectin 4 can be used to follow patients during their disease: For this purpose, we selected patients presenting increased levels of CEA or CA15.3 during disease progression. As exemplified in FIG. 5, high serum levels of Nectin 4 and CA15.3 were detected in a patient concomitantly with the appearance of pulmonary metastasis 32 months after diagnosis (gray bars). Thirty nine months after diagnosis, this patient presented cerebral metastases concomitant with a general deterioration of his health. Whereas CA15.3 slightly increased, circulating Nectin 4 levels doubled indicating that, at least in this patient, Nectin 4 was the best marker (black bars).

Nectin 4 serum detection could appear specific for breast cancer. Indeed, circulating Nectin 4 was found in 25% of sera (n=23) from patients with lung cancer (FIG. 6). In contrast the CYFRA-21 marker was detected in 65% of cases. Circulating Nectin 4 was found in 4% of sera from patients with ovary cancer. Nectin 4 was not found in all sera derived from patients with prostate cancer.

Altogether our results emphasize that Nectin 4 is a new valuable marker for metastatic breast cancer and open new alternatives in patients that present undetectable levels of CEA and CA15-3.

### DISCUSSION

We report here the characterization of a new molecular marker in breast cancer named Nectin 4. Nectin 4 is expressed during development and is repressed in adult tissues. This classifies Nectin 4 as a tumor marker that belongs to the class of embryonic antigen, like CEA or alpha-feto proteins. Nectin members are localized at adherens junctions with E-cadherin. Interestingly, Nectin 4 and E-cadherin are prominently expressed in infiltrating ductal carcinomas but much less in lobular carcinomas. In this study, 75% of the lobular carcinomas (in situ or infiltrating) have lost the cell membrane E-cadherin expression. Nectin 4 expression is strongly correlated to E-cadherin expression. Simultaneous analysis of both markers may improve the accuracy of diagnostic. In some cases, E-cadherin was found expressed in the absence of Nectin 4 expression suggesting that E-cadherin may be correctly expressed without the expression of Nectin 4. Recently, it has been suggested that the Nectin/AF-6 system may recruit the E-cadherin/catenins complex during the formation of adherens junction in epithelial cells (10). Our results suggest that E-cadherin expression is not dependent of Nectin 4 expression. Nevertheless, E-cadherin expression might be regulated by other members of the Nectin family largely expressed in all the tumor cell lines tested (data not shown). Even though the expression of the different Nectins has not been evaluated in tumors, the consequence of Nectin 4 re-expression to the tumor behavior is unclear. Analyzes of two patients with ductal carcinomas revealed expression of Nectin 4 in both primary and metastatic tumors (data not shown). This data may indicate that, in these cases, expression of Nectin 4 favors tumor progression. As most of E-cadherin expressing carcinoma cells express Nectin 4 (ductal type), it is conceivable that, at least in these cells, “illegitimate” expression of Nectin 4 contributes to disrupt cell polarity. We found a cytoplasmic localization of Nectin 4 as described for E-cadherin (24). The significance of this localization is unclear but may affect the distribution of key factors involved in cell polarity.

Circulating form of Nectin 4 is detected in 51% of sera from patients with metastatic breast cancer at diagnosis. This percentage is slightly lower to the percentage found with CEA and CA15.3. Interestingly, circulating Nectin 4 is found in patient sera negative for both CEA and CA15.3 markers. Thus Nectin 4 detection may improve the follow-up of patients during therapeutic phases. Association of the three markers allows the follow-up of 84% patients at the time of diagnosis. We noted a concomitant appearance of Nectin 4 with CA15.3 marker during disease progression suggesting that Nectin 4 may be helpful to the following the course of patients with breast cancer (FIG. 5). Of course, additional analyses will be necessary to evaluate the sensitivity of this new marker, especially its ability to anticipate the appearance of metastases. From an economic point of view, it is of note that the concomitant use of the three markers is not necessary during the follow-up of patients. Thus, we propose a protocol analyzing the three markers in a first intention, then, if possible, a subsequent selection of one or maximum two positive markers to continue the follow-up of patients.

No soluble form of Nectin 4 resulting from alternative splicing is described in EST database, suggesting that soluble Nectin 4 may result from cell surface shedding. Indeed, we found that Nectin 4 is processed by a member of the ADAM family named TACE/ADAM-17. This protease is involved in numerous shedding processes both in normal and pathological situations (25). Interestingly, TACE expression is high in breast tumors suggesting that this protease plays an important role in the biology of breast neoplasm as recently suggested (26). Recently, shedded form of Nectin-1 has been described in vitro. To date no data are available concerning the presence of circulating form of Nectin-1 in sera as well as the protease involved in this shedding.

This study highlights that Nectin 4 is a new marker for breast carcinoma. Interest of this marker also resides in its specificity. Indeed, the analysis of Nectin 4 expression in different tumor cell lines and of circulating Nectin 4 in other neoplasms, shows that Nectin 4 would be a breast specific marker. In conclusion, this marker is useful to assess the origin of a metastatic tumor at the time of diagnosis.

Nectin 4 is a new embryonic tumor antigen that brings new alternatives in the follow-up of patients with breast cancer. Furthermore, Nectin 4 can now be considered as a target for “therapeutic” antibodies and/or as an immunogen for the development of cancer vaccine-based therapies.

### EXAMPLE 2

The study is related to 109 new cases of patients with breast cancer in metastatic phase and aims at:
confirming the presence of Soluble Nectin 4 (SN4) in the patients with metastatic evolution
evaluating the advantage of this marker compared to markers CEA and CA15-3
evaluating if SN4 can be an indicator of therapeutic follow-up

The results show that SN4 is found in 33% of the patients against 52% and 47% for CEA and CA 15.3. Thus, SN4 detects less case than the two other markers. However, SN4 is detected in 4 cases out of 26 patients CEA−/CA15-3− (11%), 6 cases out of 17 of patients CEA−/CA15-3+ (35%) and 5 cases out of 23 of patients CEA+/CA15-3− (22%). These results confirm that SN4 improves follow-up in the case of patients negative for one and especially two markers. Thus, SN4 is a complementary marker to CEA and CA15-3.

The study of the evolution of SN4 before and after treatment has been carried out on 60 patients out of 109. For the 49 other patients, only one sample was available. 23 out of 60 are positive for SN4.

The table below, represents the triple-positive cases, and shows that SN4 closely follows the evolution of the 2 other markers. Clinical status was generally in accordance with the evolution of the three markers.

These results show that SN4 is a therapeutic indicator of follow-up and that its presence in sera is directly related to the development of the tumor.

TABLE 1
triple positive patients

| | | sN4 | CEA | CA15.3 | Clinical status |
|---|---|---|---|---|---|
| | P1 | [symbol] | [symbol] | [symbol] | response |
| | P2 | [symbol] | [symbol] | [symbol] | response |
| | P3 | [symbol] | [symbol] | [symbol] | stable |
| {open oversize parenthesis} | P4 | [symbol] | [symbol] | [symbol] | response |
| | P5 | [symbol] | [symbol] | [symbol] | response |
| | P6 | [symbol] | [symbol] | [symbol] | response |
| | P7 | [symbol] | [symbol] | [symbol] | response |
| | P8 | [symbol] | [symbol] | [symbol] | response |
| | P9 | [symbol] | [symbol] | [symbol] | progression |
| {open oversize parenthesis} | P10 | [symbol] | [symbol] | [symbol] | progression |
| | P11 | [symbol] | [symbol] | [symbol] | progression |
| | P12 | [symbol] | [symbol] | [symbol] | progression |
| {open oversize parenthesis} | | | | | |
| | P13 | [symbol] | [symbol] | [symbol] | progression |

LEGEND: [symbol]
negativity [symbol]
decrease [symbol]
stable [symbol]
positivity [symbol]
increase

Table 2 summarizes the positive patients for SN4 and one of the two other markers or for SN4 only. This study is interesting because it integrates at the same time the concept of “complementary marker” and “marker of therapeutic efficiency”.

TABLE 2
double and simple positive patients

| | sN4 | CEA | CA15.3 | Clinical status |
|---|---|---|---|---|
| P14 | [symbol] | — | [symbol] | response |
| P15 | [symbol] | — | [symbol] | progression |
| P16 | [symbol] | — | [symbol] | response |
| P17 | [symbol] | [symbol] | — | response |
| P18 | [symbol] | [symbol] | — | response |
| P19 | [symbol] | [symbol] | — | response |
| P20 | [symbol] | [symbol] | — | response |
| P21 | [symbol] | — | — | progression |
| P22 | [symbol] | — | — | response |
| P23 ^{#} | [symbol] | — | — | stable |

To conclude, this new study confirms that SN4 is a new promising serum marker in metastatic breast cancer and highlights new interesting points:
SN4 can be a “complementary marker” in CEA− and CA15.3− patients.
SN4 strengthens the follow-up of patients having one of the two markers.
Evolution of the rates of SN4 before and after similar treatment that of the two other markers. SN4 is a reliable marker of therapeutic efficiency for breast carcinomas.
   # P23: Patient under continuous evaluation.

### REFERENCES

1. Sidransky, D. (2002) Nat Rev Cancer 2, 210-9.
2. Gold, P. & Freedman, S. O. (1965) J Exp Med 122, 467-81.
3. Tobias, R., Rothwell, C. & Wagner, J. (1985) Proceedings of the Symposium of the AACC, Atlanta.
4. Oncology, A. S. o. C. (1996) J. Clin. Oncol. 14, 2483-2877.
5. Lopez, M., Eberle, F., Mattei, M. G., Gabert, J., Birg, F., Bardin, F., Maroc, C. & Dubreuil, P. (1995) Gene 155, 261-5.
6. Eberle, F., Dubreuil, P., Mattei, M. G., Devilard, E. & Lopez, M. (1995) Gene 159, 267-72.
7. Reymond, N., Borg, J. P., Lecocq, E., Adelaide, J., Campadelli-Fiume, G., Dubreuil, P. & Lopez, M. (2000) Gene 255, 347-55.
8. Reymond, N., Fabre, S., Lecocq, E., Adelaide, J., Dubreuil, P. & Lopez, M. (2001) J Biol Chem 276, 43205-15.
9. Mendelsohn, C. L., Wimmer, E. & Racaniello, V. R. (1989) Cell 56, 855-65.
10. Takai, Y. & Nakanishi, H. (2003) J Cell Sci 116, 17-27.
11. Lopez, M., Aoubala, M., Jordier, F., Isnardon, D., Gomez, S. & Dubreuil, P. (1998) Blood 92, 4602-11.
12. Belaaloui, G., Imbert, A. M., Bardin, F., Tonnelle, C., Dubreuil, P., Lopez, M. & Chabannon, C. (2003) Leukemia 17, 1137-45.
13. Cocchi, F., Menotti, L., Mirandola, P., Lopez, M. & Campadelli-Fiume, G. (1998) J Virol 72, 9992-10002.
14. Reymond, N., Imbert, A. M., Devilard, E., Fabre, S., Chabannon, C., Xerri, L., Farnarier, C., Bottino, C., Dubreuil, P., Moretta, A. & Lopez, M. (2004) J. Exp. Med. In press.
15. Fabre, S., Reymond, N., Cocchi, F., Menotti, L., Dubreuil, P., Campadelli-Fiume, G. & Lopez, M. (2002) J Biol Chem 277, 27006-13.
16. Suzuki, K., Hu, D., Bustos, T., Zlotogora, J., Richieri-Costa, A., Helms, J. A. & Spritz, R. A. (2000) Nat Genet 25, 427-30.
17. Kim, D. Y., Ingano, L. A. & Kovacs, D. M. (2002) J Biol Chem 277, 49976-81.
18. Tanaka, Y., Irie, K., Hirota, T., Sakisaka, T., Nakanishi, H. & Takai, Y. (2002) Biochem Biophys Res Commun 299, 472-8.
19. Marin, V., Kaplanski, G., Gres, S., Farnarier, C. & Bongrand, P. (2001) J Immunol Methods 254, 183-90.
20. Ginestier, C., Charafe-Jauffret, E., Bertucci, F., Eisinger, F., Geneix, J., Bechlian, D., Conte, N., Adelaide, J., Toiron, Y., Nguyen, C., Viens, P., Mozziconacci, M. J., Houlgatte, R., Birnbaum, D. & Jacquemier, J. (2002) Am J Pathol 161, 1223-33.
21. De Leeuw, W. J., Berx, G., Vos, C. B., Peterse, J. L., Van de Vijver, M. J., Litvinov, S., Van Roy, F., Cornelisse, C. J. & Cleton-Jansen, A. M. (1997) J Pathol 183, 404-11.
22. Jacquemier, J., Eisinger, F., Nogues, C., Sun, Z. Z., Guinebretiere, J. M., Peyrat, J. P., Geneix, J., Lidereau, R., Birnbaum, D. & Sobol, H. (1999) Int J Cancer 83, 45-9.
23. Sarrio, D., Moreno-Bueno, G., Hardisson, D., Sanchez-Estevez, C., Guo, M., Herman, J. G., Gamallo, C., Esteller, M. & Palacios, J. (2003) Int J Cancer 106, 208-15.
24. Gillett, C. E., Miles, D. W., Ryder, K., Skilton, D., Liebman, R. D., Springall, R. J., Barnes, D. M. & Hanby, A. M. (2001) J Pathol 193, 433-41.
25. Black, R. A. (2002) Int J Biochem Cell Biol 34, 1-5.
26. Borrell-Pages, M., Rojo, F., Albanell, J., Baselga, J. & Arribas, J. (2003) Embo J 22, 1114-24.
27. Lopez, M. Jordier, F. et al in leucocytes typing VI: White Cell Differenciation Antigens (Kishimoto, T., Kikutani, H., von dem Borne, A. E. G., Goyert, S. M., Mason, D. Y., Miyasaka, M., Moretta, L., Okumura, K., Shaw, S., Springer, T. A., Sugamura, K., Zola, H. eds) pp. 1081-1083, Garland Publishing, New York
28. Takahashi, K., Nakanishi, H., Miyahara, M et al (1999) J. Cell Biol. 145, 539-549
29. Geraghty, R et al (1998) Science 280, 1618-1620
30. Warner, M. S. et al (1998) Virology 246, 179-189;
31. Lopez, M. Cocchi, F. et al (2000) J. Virol. 74, 1267-1274
32. Cocchi F., Menotti, L. et al (2000) J. Virol. 74, 3909-3917
33. Zhadanov, A. B., Provance, D. W. et al (1999) Curr. Biol. 9, 880-888
34. Ikeda, W., Nakanishi II., et al (1999) J. Cell. Biol. 146, 1117-1132
35. Harlow, E., and D. Lane. 1988. Antibodies A Laboratory Manual. Cold Spring Harbor Laboratory. pp. 53-242
36. M. Schena, D. Shalon, R. W. Davis, and P. O. Brown, “Quantitative monitoring of gene expression patterns with a complementary DNA microarray,” Science, 270 (5235), 467-70, 1995
37. Kononen et al. Nat Med. 1998 July; 4 (7):844-7
38. Schraml et al. Clin Cancer Res. 1999 August; 5 (8):1966-75
39. Bubendorf et al. Cancer Res. Feb. 15, 1999 (4):803-6
40. Bubendorf et al. J Natl Cancer Inst. Oct. 20, 1999; 91 (20):1758-64
41. Bowen et al. Cancer Res. Nov. 1, 2000; 60 (21):6111-5
42. Petricoin F, Use of proteomic patterns in serum to identify ovarian cancer, The Lancet, Vol. 359, Feb. 16, 2002
43. Zhao Rui et al, Use of serological proteomic methods to find biomarkers associated with breast cancer, Proteomics, Vol. 3, Issue 4, p 433-439, 2003.
44. Sandy Kennedy, Toxicology Letters 120 (2001) 379-384
45. Clementi et al., Competitive polymerase chain reaction and analysis of viral activity at the molecular level. GATA 1994; 11:1-6
46. Kahn et al., Neurosci. Lett. 1992; 147:114-117
47. Becker-Andre, Quantitative Evaluation of mRNA levels. Meth. Molec. Cell Biol. 1991 2:189-201.
48. Gibson et al., A novel method for real time quantitative RT-PCR Genome research. 1996; 6:995-1001
49. Heid et al., Real-time quantitative PCR. Genome Research. 1996; 6:986-994; and Livak et al.,

1. An in vitro or ex vivo method for prognosing cancer comprising detecting the presence or the absence of Nectin 4 in a sample, the presence of Nectin 4 being indicative of cancer.

2. The method according to claim 1 wherein Nectin 4 is detected by at least one anti-Nectin 4 antibody.

3. The method according to claim 1 wherein the cancer is a metastatic cancer.

4. The method according to claim 3 wherein the cancer is a breast cancer.

5. The method according to claim 1 wherein the sample is a subject's serum or plasma.

6. The method according to claim 5, wherein Nectin 4 is on a soluble form.

7. The method of claim 6 which further comprises dosing the level of soluble Nectin 4.

8. The method of claim 7 wherein the presence of at least 30 pM of soluble Nectin 4 is indicative of a metastatic cancer.

9. The method according to claim 2 wherein said anti-Nectin 4 antibody is directed against the soluble form of Nectin 4.

10. The method according to claim 9 wherein said anti-Nectin 4 antibody is directed against the ectodomain of Nectin 4.

11. The method according to claim 5, wherein the presence of soluble Nectin 4 is determined by Immuno Assay.

12. The method according to claim 11, wherein the immuno assay is an Enzyme-Linked Immuno Sorbent Assay (ELISA).

13. The method according to claim 1, wherein the sample is subjects' tumor cells.

14. The method of claim 13 which further comprising detecting the signal and determining the ratio of cell-expressing Nectin 4.

15. The method of claim 14 wherein a quick score (percentage of cells expressing Nectin 4× labeling intensity) above 10 is indicative of a cancer.

16. The method according to claim 13, wherein the presence of Nectin 4 is determined by immunohistochemistry.

17. The method according to claim 2, wherein said antibody is a monoclonal or polyclonal antibody or a Fab or a F(ab′)2 fragment thereof.

18. The method according to claim 2, wherein it comprises the use of a labeled anti-Nectin 4 antibody.

19. The method according to claim 2, wherein it comprises the use of an anti-Nectin 4 primary antibody and a staining with a labeled second reagent antibody directed against said primary antibody.

20. The method according to claim 1, comprising besides detecting the presence or the absence of other cancer markers.

21. A method for medical imaging a tumor from a subject comprising:
a) incubating the tumor with a labeled anti-Nectin 4 antibody for a sufficient period of time to permit the antibody to react with Nectin 4;
b) detecting the presence of the label localized to the tumor.

22. A method for prognosis cancer comprising detecting Nectin 4 level in a subject sample and determining the level of Nectin 4 compared to the level in a control sample, a significant level in a subject sample being indicative of a poor outcome.

23. A method for the therapeutic follow-up of an anticancer treatment of a subject characterized in that the presence or the absence of Nectin 4 is detected during or after the treatment.

24. The method according to claim 23, wherein the absence or the decrease of Nectin 4 is significant of a positive response to the treatment.

25. The method according to claim 22 wherein the sample is a subject's serum.

26. The method according to claim 22 wherein Nectin 4 is on a soluble form.

27. The method according to claim 22, wherein the sample is subject's tumor cells.

28. A kit for performing the method as defined in claim 1, comprising either a labelled anti-Nectin 4 or a first anti-Nectin 4 antibody and a second labelled anti-Nectin 4 antibody.

29. Kit comprising a first anti-Nectin 4 antibody and a second anti-Nectin 4 antibody, said first and second antibody being directed against different Nectin 4 epitopes and wherein the binding of the first antibody does not interfere with the binding of said second antibody.

30. The kit as defined in claim 28 for the prognosis of subjects afflicted with cancer and the initiation of adequate therapy early in the cause of the disease.

31. The kit according to claim 30 for providing an ex vivo assessment of the antitumor effects of the chemotherapy in the course of the therapy.

32. The kit as defined in claim 28 for use as a predictor of cancer prognosis and survival.

33. The method of claim 8 wherein the metastatic cancer is a metastatic breast cancer.

34. The method of claim 15 wherein the cancer is a metastatic cancer.

35. The method of claim 34 wherein the cancer is a metastatic breast cancer.

36. The method of claim 20 wherein other cancer markers are carcinoma embryonic antigen (CEA) and CA 15-3.

37. The kit according to claim 32 wherein the cancer is a breast cancer.

38. The method according to claim 1 for the prognosis of subjects afflicted with cancer and the initiation of adequate therapy early in the cause of the disease.

39. The method according to claim 1 for providing an ex vivo assessment of the antitumor effects of chemotherapy during the course of the therapy.

40. The method according to claim 1 for use as a predictor of cancer prognosis and survival.
